(19) United States
(12) Patent Application Publication
(10) Pub. No.: US 2005/0267569 A1
(43) Pub. Date: Dec. 1, 2005

(54) Non-invasive detection of in-stent stenosis and drug elution

(76) Inventors: Gary Barrett, Alpharetta, GA (US); Jeffrey Wallace, Red Bank, NJ (US); Nitish V. Thakor, Clarskville, MD (US); Ananth L. Natarajan, San Marino, CA (US)

Correspondence Address: Ananth Natarajan, MD, Suite 410, 3600 Clipper Mill Road, Baltimore, MD, 21211, US

(21) Appl. No.: 11/089,625

(22) Filed: Mar. 25, 2005

Related U.S. Application Data
(60) Provisional application No. 60/556,990, filed on Mar. 27, 2004.

(51) Int. Cl. A61F 2/06

(52) U.S. Cl. 623/1.44

(57) ABSTRACT

Devices and methods are described. One apparatus includes a stent including a sensor coupled thereto, the sensor including a material that oscillates when subjected to an applied magnetic field. The material may include at least one material selected from magnetoelastic materials and magnetorestrictive materials. The apparatus may further include a system adapted to monitor the sensor, the system including a generator adapted to apply a magnetic field that generates physical oscillations in the sensor, and a monitor adapted to detect magnetic fluctuation generated from the physical oscillations in the sensor. Other embodiments are described and claimed.

This application claims priority in U.S. Provisional Application No. 60/556990, filed Mar. 27, 2004, which is hereby incorporated by reference in its entirety.

### FIELD OF INVENTION

Certain embodiments relate to methods and devices for detection and monitoring of conditions in a patient. Such conditions may include, but are not limited to, in-stent stenosis thrombosis, platelet accretion, coronary artery disease, drug elution, drug delivery, blood viscosity changes and variations in the physical nature of bodily fluids as an indication of one or more of illness, disease, medical conditions, and injury.

### BACKGROUND

Stenosis is a narrowing, or complete occlusion of an artery, which reduces perfusion of the tissues supplied by that artery. Stents are often introduced via angioplasty to open these vessels. Restenosis is the development of occlusion as a result of damage to the arterial vessel epithelium as a result of mechanically or chemically induced irritation of the arterial wall. The imposed damage to the artery results in a series of biochemical and biomechanical actions leading to localized inflammation, thrombus formation, and potentially to total arterial occlusion. Platelets and white blood cells from the blood migrate into the injured intima (inner layer of the vessel). The release of cytokines and T-lymphocytes stimulate smooth muscle cells (cells from the wall of the artery) to migrate and divide, in an attempt to repair the wound. This process is enabled by the white blood cells releasing and activating tissue-digesting enzymes, forming a path for the smooth muscle cells to move. The inflammatory response results in localized scar tissue development, and in the case of stent application this may lead to the development of thrombus formation, plaque deposition and neo-intimal tissue generation encompassing the stent and leading to occlusion of the artery with a corresponding impact on function. The redevelopment of an occlusion significantly affects medical outcome and increases the likelihood of subsequent stenosis, cardiac infarction, and other negative outcomes. Restenosis may be defined as the presence of >50% diameter stenosis in the dilated segment of the arterial vessel targeted for treatment.

The occurrence of restenosis following interventional therapy has been a subject of research effort since the inception of stent therapy. Current methods and fabrications of stents include bare metal, polymer coated metal, shape memory alloy, brachyotherapy and drug eluting methodologies amongst others. The primary goal of the research and development efforts has been to combat the occurrence of restenosis and to increase arterial flow for a prolonged period. While various methods have been shown to reduce the likelihood of restenosis after angioplasty, the number of cases is still significant. Additionally; several therapies, while reducing the likelihood of restenosis, have secondary effects. For example, in certain cases, drug eluting stents have been shown to instigate secondary irritation of the arterial wall, leading to vascular damage and increased scarring.

Diagnosis of restenosis incidence is currently performed from symptomatic chest pain. Confirmation is obtained using cardiac stress tests and arterial catheterization.

Thrombosis is a localized coagulation response mitigated by myriad biochemical and physiological responses. Thrombus development has been observed following the application of certain drug eluting stents. While anti-platelet therapy has been found to reduce the occurrence of thrombus within the stent, the long term implications of drug eluting stents have yet to be fully characterized. Thrombus formation leads to arterial occlusion resulting in the development of myocardial ischemia and other potentially fatal conditions including stroke.

### BRIEF DESCRIPTION OF THE DRAWINGS

Certain embodiments of the invention are described with reference to the accompanying drawings, which, for illustrative purposes, are not necessarily drawn to scale.

FIGS. 1(a)-(b) illustrate schematics of arterial occlusion, requiring angioplasty or other intervention.

FIGS. 2(a)-(b) illustrate schematics of arterial occlusion with the application of a stent.

FIGS. 3(a)-(e) illustrate schematics showing various forms of in stent restenosis.

FIG. 4 illustrates a schematic of magnetic excitation and remote pick-up system for interrogation of magnetoelastic or magnetorestrictive strip or wire for amplitude and frequency oscillation in accordance with certain embodiments of the present invention.

FIG. 5 illustrates a schematic showing changes in resonance frequency due to parameter changes in accordance with certain embodiments of the present invention.

FIG. 6(a)-(c) illustrate a schematic of a Helmholtz coil system to excite vibrations in, and graphs to measure signals resulting from these vibrations in a strip or wire made of magnetoelastic or magnetorestrictive material in accordance with certain embodiments of the present invention.

FIG. 7 illustrates a schematic of data transmission and detection in accordance with certain embodiments of the present invention.

### DETAILED DESCRIPTION

The development of restenosis, thrombosis and arterial lumen encroachment have been identified as biochemical and physiological responses to the application of stents, drug eluting stents and arterial angioplasty interventions.

Certain embodiments of the present invention are designed to permit the identification, characterization and assessment of a variety of forms of arterial occlusion.

Such embodiments of the present invention may be designed for the monitoring of occlusion related to coagulation and plaque deposition within the stent and within the immediate vicinity of the stent. In certain embodiments, detection may be carried out using an external source using the monitoring of magnetic or sound waves generated by alteration of oscillatory frequency of at least a portion of the material that may be incorporated into sensor. Such embodiments may be used to monitor, diagnose and interrogate coagulation, plaque deposition, alteration in viscosity, temperature, pressure and flow rate of liquids including, but not limited to, blood, lymph, plasma, lachrymal secretions, cerebro-spinal fluid, semen and bodily secretions.

Certain embodiments of the present invention may be incorporated into stents already incorporating anti-restenosis therapies and used in conjunction with anti-restenosis therapies including, but not limited to; coated stents, drug release stents (heparin etc), drug eluting stents, brachyotherapy, platelet aggregation inhibitors, GP IIb/IIIa inhibitors; antiproliferative growth factor inhibitors, and statins and lipid-lowering agents.

Certain embodiments will allow the monitoring of restenosis and other occlusions using a portable unit, or a static monitoring system. Embodiments may be integrated within stents to allow the monitoring of arterial occlusion formation over time, and the chemical/physical identity of the plaque may be identified as a function of mass loading and viscosity. Certain embodiments may also monitor the rate and mass of drugs released from drug eluting stents and may perform long term monitoring of arterial scarring, plaque formation and thrombus formation. Additionally, certain embodiments may be attached to extend beyond the stent boundaries to detect restenosis, stenosis and arterial deposits outside of the immediate boundary of the stent.

Certain embodiments are based on magnetoelastic/magnetorestrictive or similar materials conferring an oscillatory response as a proportional response to changes in the local environment or to changes that impinge on the environment of the sensor. While the description presented below concentrates primarily on the applicability of magnetoelastic materials, it should be appreciated that there may be other materials that may also be used.

Certain embodiments are intended for one or more of the diagnosis, identification, assessment, monitoring and evaluation of conditions including, but not limited to, stenosis, restenosis, thrombosis, platelet accretion and arterial rebound. The sensor may be introduced to an arterial stent or suspended independently within blood vessels or body cavities to measure blood or body fluid physical, physicochemical and physiological properties.

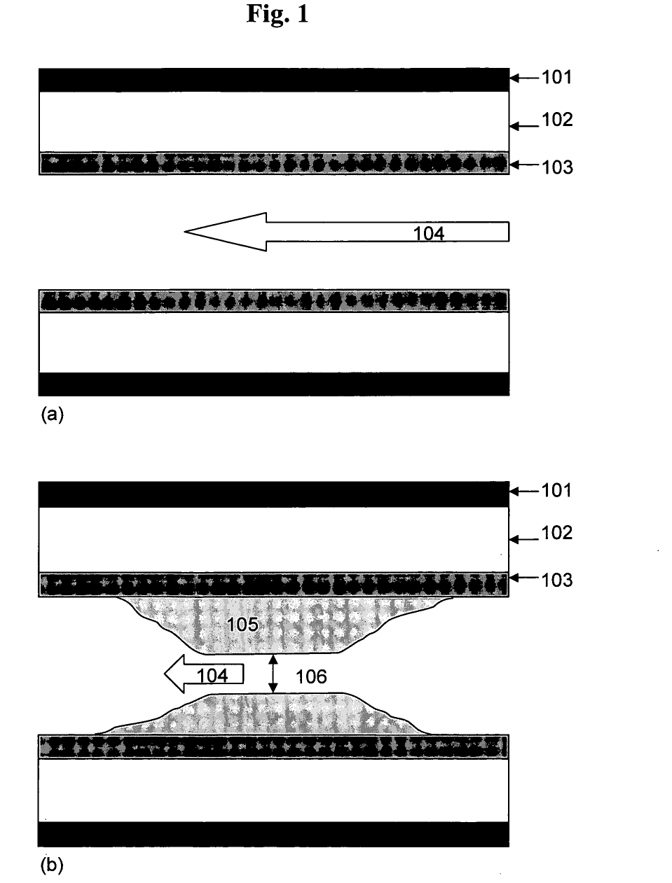

Coronary artery disease, stenosis and arterial occlusion may be treated with the application of arterial stents. Stent insertion is a minimally invasive method of opening the internal diameter of occluded arteries, allowing increased blood flow. As illustrated in FIGS. 1(a)-(b), in the development of an arterial occlusion, the arterial lumen (104) is occluded by the generation of an arterial plaque (105) impinging arterial blood flow (106) and causing a significant narrowing of the arterial lumen. The arterial wall structure (101—Collagen and elastic; 102 Media and intima—smooth muscle layer and 103 Basal Epithelia) may develop significant biochemical responses, leading to arterial wall rebound.

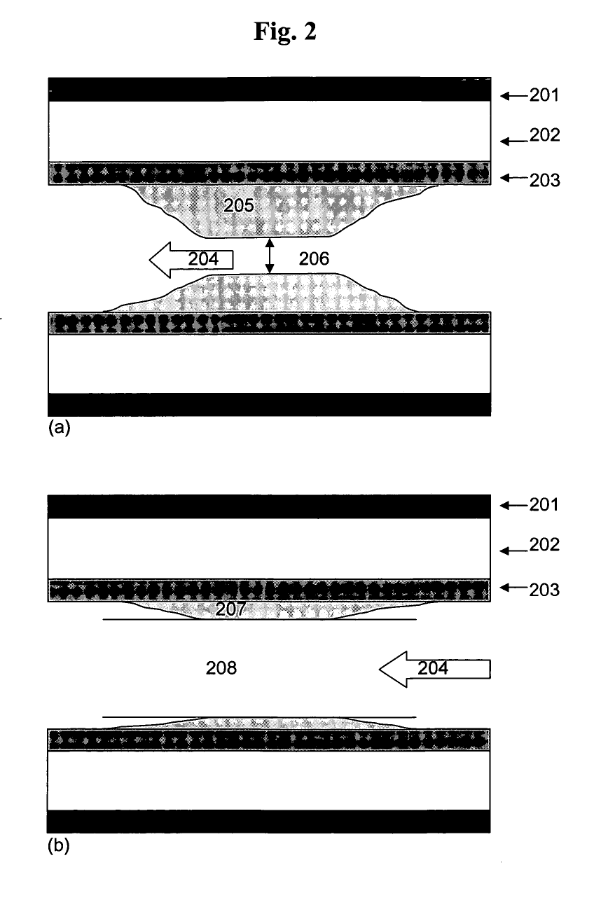

As illustrated in FIGS. 2(a) and 2 (b), the application of a stent (208) can be seen to open the occlusion (205 in FIG. 2(a), 207 in FIG. 2(b)), thus increasing the size (206) of the arterial lumen and allowing better blood flow (204). The plaque or occlusion (205), (207) is pushed into the arterial wall (201, 202, 203).

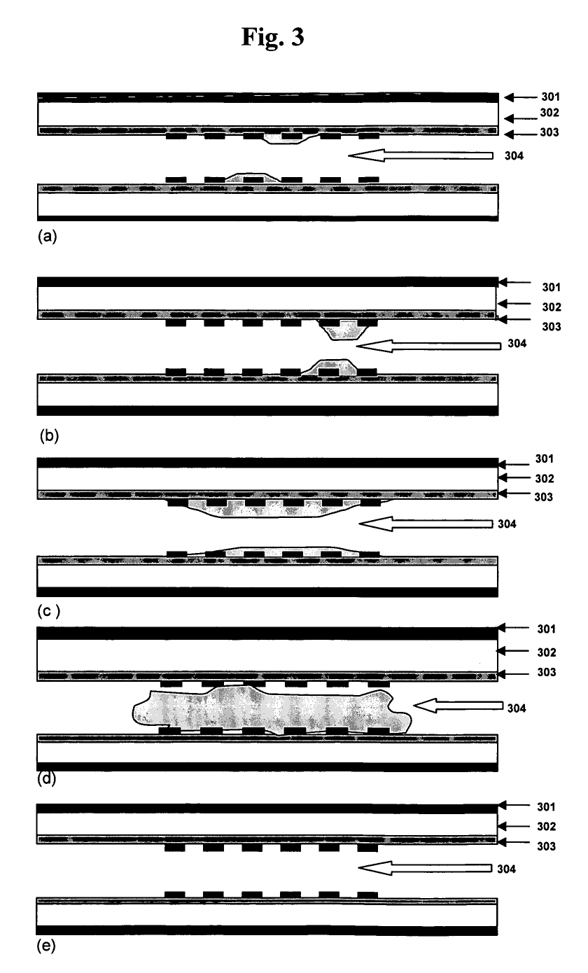

FIGS. 3(a)-(e) shows some of the effects of restenosis. The impact of the stents expansion into the arterial wall (301—Collagen and elastic; 302 Media and intima—smooth muscle layer and 303 Basal Epithelia), may cause a biochemical response, resulting in the release of cytokines and other endothelial irritants, resulting in the accretion of platelets, arterial rebound and plaque formation. These issues may result in a renarrowing of the artery and the development of a restenosis. The restenosis may be focal (FIG. 3a) and (FIG. 3b), diffuse—expanding the length of the stent (FIG. 3c), or a total occlusion (FIG. 3d). Additionally, the occlusion may be within the borders of the stent or proliferative where the occlusion expands beyond the stent periphery, where it impacts the arterial wall. FIG. 3e shows an embodiment where no occlusion is present in the arterial lumen.

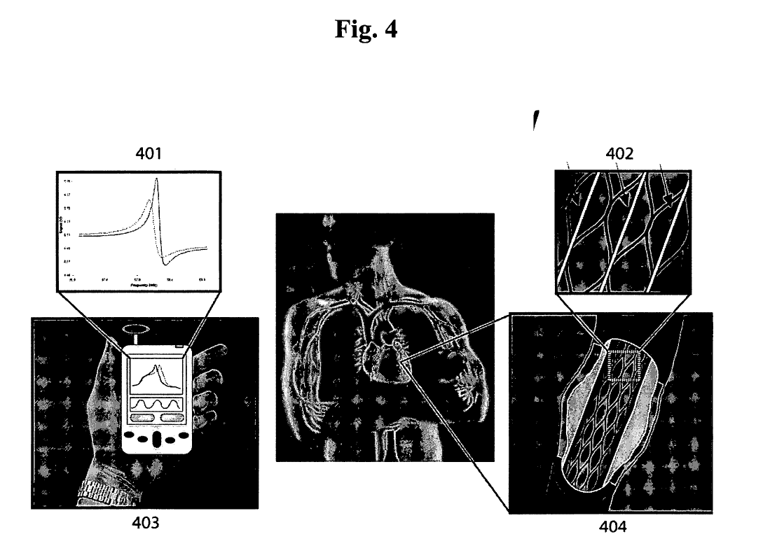

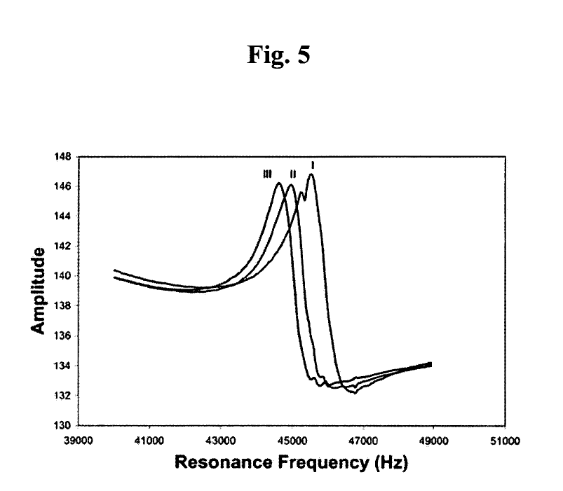

Certain embodiments include a sensor system aimed at the monitoring, diagnosis and evaluation of restenosis, thrombosis and other cardiac events using a remote monitoring system, as illustrated in FIG. 4. By integration of a sensor wire or strip into or attached to the stent (402), the stent may be remotely monitored (403), allowing a real time analysis of the local environment of the stent (401). Other sensor forms in addition to wire and strip forms are possible, including, but not limited to, tubular forms or plate forms. FIG. 5 shows an embodiment where an externally applied magnetic field excites a characteristic longitudinal oscillation in a magnetoelastic sensor. The resonant frequency alters as a function of mass loading on the sensor surface, and the corresponding resonate peaks (I, II, III) show real time alteration of the sensor as a function of impinged mass.

The magnetoelastic or magnetorestrictive material of certain embodiments are amorphous metal alloys of either a cobalt, ferrous or nickel base with varying metallic additions to alter physical properties. Certain embodiments may be coated with biocompatible coatings, as well as drug eluting systems and other drug delivery systems. Embodiments may include a sensor system that is incorporated into a stent frame or suspended from a stent by connections at the point/s of harmonic oscillation. Additionally, certain embodiments may include a sensor system that is attached using elastomeric compounds, adhesives or other physical or chemical connection systems, to allow free oscillation.

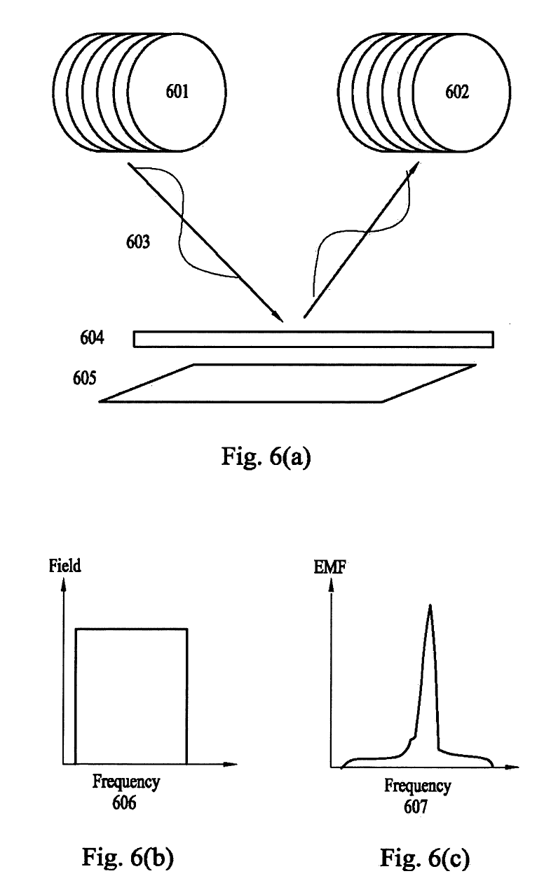

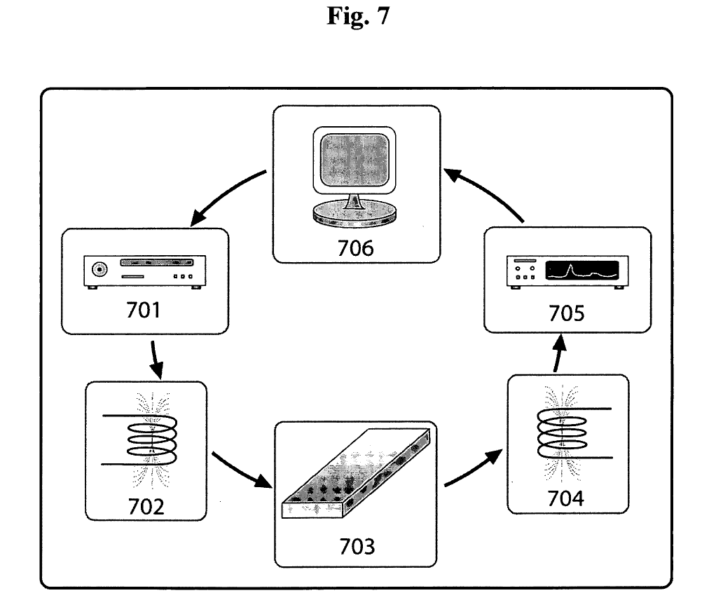

When exposed to magnetic fields, as illustrated in FIGS. 6(a)-(c), magnetoelastic materials (wires 604 and strips 605) oscillate in a longitudinal fashion. The frequency and amplitude of the oscillation is dependant on the physical parameters of the material (length, width and thickness) as well as the physical parameters of the local environment. Excitation from a remote magnetic source (601) may be monitored using a detection system (602), either as part of the excitation system or as a remote unit. In the illustrated embodiment, the excitation magnetic source (601) creates a magnetic field (606) that causes oscillations in the magnetoelastic materials. The resonant frequencies of the oscillations (607) may be detected by the detection system (602). Certain embodiments can monitor resultant oscillations as a change in magnetic field strength, electromotive force, or others. Embodiments may be used to monitor viscosity, pressure and physical encroachment/mass loading, allowing identification of various effects by characteristic alteration of sensor response to excitation. Thus, coagulation leading to a thrombus formation will be differentiated from the build-up of a calcified plaque and/or arterial wall encroachment. Additionally, the oscillation generates a sound wave with a characteristic frequency and amplitude; again these factors are determined by the local environment. Thus, changes in frequency and amplitude can be monitored to allow interrogation of the embodiment environment where, for example, detection coils can be replaced using high quality microphone systems. FIG. 7 illustrates an embodiment including a power supply (701) for powering an activating coil (702). The activating coil (702) provides a magnetic field to the magnetoelastic sensor (703). The magnetoelastic sensor (703) vibrates at a frequency in part based on the conditions on and adjacent to the sensor. The vibrations are detected by the sensing coil (704), and transmitted to a spectrometer (705) for evaluation. Aspects of the system may be controlled and monitored using a computer (706). By monitoring any changes to the frequency over time, any changes in the conditions on and adjacent to the sensor (coupled to the stent) can be detected and an appropriate response (therapy, etc.) undertaken. The types of conditions that may be monitored or evaluated include, but are not limited to stenosis, thrombosis, coronary artery disease, changes in blood viscosity, platelet accretion, physiological parameters, non-physiological parameters, and drug delivery.

Magnetoelastic sensors generate an electromotive force when exposed to a magnetic field; it is this force and generated oscillation that is detected via the pick-up coil in certain embodiments. The electromotive force results in physical longitudinal oscillation. Magnetorestrictive sensors change their characteristic resistance, and hence the frequency at which they resonate when excited. These oscillations have a resonant frequency (the frequency of oscillation that results in maximum harmonic motion), which is altered by perturbation in the physical environment, the chemical environment, or the physiologic environment, or physical nature of the sensor embodiment itself (length, thickness etc). A comparable analogy is the harmonic generation of sound from a crystal wine glass, in which the frequency of the sound generated alters on the addition of water to the glass. As certain embodiments are dependant on a magnetic field generation for both excitation and detection, no physical or optical connections are necessary. This factor makes magnetoelastic material extremely attractive as an embodied sensor for biomedical applications. Embodiments as described herein may also generate a harmonic sound wave; this may be used as a monitoring system for examination of the magnetoelastic sensor.

As sensor in various embodiments may have a specific resonant frequency, several sensors of varying length may be used that can be multiplexed into a stent. The sensors may be configured to propagate from the stent body. By using sensors of different lengths, each resonant frequency can be examined individually, allowing the interrogation of the stent at all points, and permitting a three dimensional map of any occlusion developing within the stent. Interrogation of these embodied sensors in series will then allow the examination of the stent at different regions, thus allowing identification of the exact position of restenosis and/or plague build up. The sensor may thus include multiple sensors that are multiplexed into a stent, allowing the monitoring of distal and proximal ends, as well as medial restenosis, as separate signal pathways with no interference.

At the resonance frequency, the applied external ac magnetic field generates a longitudinal elastic standing wave. The ribbon's magnetic anisotropy is cancelled by a superimposed dc magnetic field. If the excitation and the characteristic resonance frequency of the embodiment are the same, maximal conversion of the magnetic energy into elastic energy is obtained, resulting in a magnetoelastic resonance. The following expression predicts the behavior of the magnetoelastic sensor by considering the longitudinal resonance frequency of a thin ribbon-like strip vibrating in its basal plane:

$${f}_{n} = \sqrt{\frac{E}{ρ ( 1 - {σ}^{2} )}} \frac{n π}{L}$$

Where E is Young's modulus of elasticity, σ is the Poisson ratio of the material, ρ is the density of the sample, L is the length of the ribbon, n and denotes positive integers describing high order harmonics. These parameters allow monitoring of local environmental conditions via changes in frequency, amplitude and evolved sound.

Certain embodiments of the present invention utilize magnetoelastic or magnetorestrictive materials in the development of a dilatory stent. Such material allows external non-contact interrogation of the stent device, allowing the detection and/or monitoring of post-stent restenosis. Magnetoelastic sensors monitor localized mass changes and variation in the physical parameters of the media in which the sensor embodiment is suspended. As such, that the sensor embodiment may in certain embodiments be used to monitor the release of drugs from eluting stents, restenotic events, thrombosis and other coagulation and thickening events within the blood, lymph and other body fluids.

In-stent restenosis generally is a relatively early event occurring shortly after interventional treatment. The condition begins on the cellular level within 72 hours of a revascularization procedure and may become more apparent on the vascular level within two weeks of the procedure. The extent of restenosis is typically measured via an angiogram or through Quantitative Coronary Angiography (QCA). The problem is exacerbated by a combination of biochemical and biomechanical effects.

The sequence of events leading to restenosis generally includes an initial mechanical injury which typically results from angioplasty techniques for delivering stents. This injury triggers localized cytokine production, which leads to a localized immune response of inflammation. Macrophages and T lymphocyte production also occur. This in turn leads to thrombus formation, intimal hyperplasia (smooth muscle cell growth in a localized area), the remodeling of the arterial wall and vessel wall recoil. The direct result of these biochemical and mechanical actions is the localized growth of muscle tissue in and around the stent device, leading to a regenerated occlusion with subsequent loss in localized blood flow. Recent developments in the application of stent devices include development of drug eluting stents. These embodiments are standard stent frameworks with a polymer coating added. The polymer coating is doped with anti-inflammatory and/or other drugs (specific drugs vary with the manufacturer). The slow release of the drug from the polymer coated stent directly affects cytokine release in the artery. Thus, the biochemical development of restenosis is reduced. While the application of drug eluting stents has been successfully identified as reducing restenosis by a significant factor, the long term implications of polymer coated stents have not been clearly identified. Additionally, the application of drug eluting stents has been correlated with the development of potentially fatal thromboses within the coronary arteries.

Certain embodiments of the present invention described herein include a sensor to detect and monitor generation of such an occlusion, allowing effective intervention. Certain embodiments detect development of localized mass encroachment, thus restenosis and localized thrombosis will be identified.

Certain embodiments of the present invention may also utilize the induced oscillation of a magnetoelastic or magnetorestrictive material to determine the onset of arterial occlusions within a coronary or peripheral arterial stent.

Certain embodiments of the present invention include a metallic alloy that is a magnetoelestic material and that may be coated with a biocompatible polymer to decrease the likelihood of autoimmune response. The coated alloy may be integrated directly into the body of the stent device or attached to the stent in such a manner as to allow the free oscillation of the material. The coated alloy (which may in certain embodiments be in strip or wire form) requires no power or direct physical connection; excitation comes from the application of a magnetic field. This may be achieved in certain embodiments using a Helmholtz coil apparatus or using a direct magnetic field level of controlled volume and uniformity. Depending on the application, Helmholtz field generation can be static, time varying, DC or AC. Here, the application is to generate a static or quasistatic magnetic field that will induce oscillations in the magnetoelastic material. The oscillation of the magnetoelastic material is detected using a pick-up coil, which may or may not be separate from the excitation coil. The signal is amplified, and the frequency and amplitude of oscillation are determined. Alternatively, the magnetoelastic material can be excited into oscillation by a non-Helmholtz magnetic field generator, as shown in FIG. 3. The drive coil generates a directed magnetic excitation causing the remote magnetoelastic material to oscillate; the pick up coil monitors the resultant oscillation as a function of the electromotive force and frequency of the oscillating strip. This allows the monitoring of magnetoelastic strips in millimeter lengths.

In addition to monitoring magnetic field, the magnetoelastic strip oscillation will result in the generation of sound waves of specific frequency and amplitude. Thus, certain embodiments of the present invention may utilize sound as a detection criteria.

In instances, the frequency and amplitude of magnetoelastic strip oscillation is dependant on the physical characteristics of the strip. Thus, varying lengths, thicknesses and surface roughness of the strip may alter the characteristic frequency. This allows incorporation of the strip into a stent device of varying lengths. An initial post-operative scan of the sensor will give an individualized baseline for future comparison. Subsequent fluctuations in the oscillation frequency and amplitude will be as a function of increased mass loading or localized pressure. This increased mass loading, or other changes in physical, physiochemical, or physiological parameters, will likely result from in-stent restenosis. As the arterial wall swells and encroaches on the stent, the sensor will be likewise encroached, resulting in baseline changes in oscillation. Likewise, if a regeneration of arterial plaque and/or platelet deposition results around the stent, this will significantly alter the baseline frequency and amplitude of the sensor embodiment.

The incorporation of several sensors into a single stent (dependent on length of stent) will allow the individualized monitoring of varying areas of the stent to identify the specific area at which restenosis develops.

The magnetoelastic material can be incorporated as a wire or metallic strip of various lengths within the millimeter to centimeter range. Addition of biocompatible coating (e.g. anti-inflammatory drugs) may reduce signal strength but will still allow the application to function.

Coronary and peripheral stents are used to increase localized blood flow through an occluded artery. The occlusion may result from mechanical damage to the arterial wall followed by localized scar tissue development. Additionally, biochemical response may lead to the development of thrombus, cytokine induced inflammation, platelet accretion and/or smooth muscle development.

Certain embodiments of the present invention are designed to measure the alteration in baseline oscillatory frequency. The alteration in frequency is a result of changes in physical, physiochemical, or physiological parameters. Thus, such embodiments will be able to detect and identify various responses to arterial disease within the stent. Embodiments of the present invention, while aimed at the detection of restenosis, should be able to clearly identify thrombus formation and arterial encroachment over time.

It is, of course, understood that modification of the present embodiments of the invention, in its various aspects, will be apparent to those skilled in the art. Additional method and device embodiments are possible, their specific features depending upon the particular application.

1. An apparatus comprising a stent including a sensor coupled thereto, the sensor comprising a material that oscillates when subjected to an applied magnetic field, wherein the material comprises at least one material selected from the group consisting of magnetoelastic materials and magnetorestrictive materials.

2. An apparatus according to claim 1, further comprising a system adapted to monitor the sensor, the system comprising a generator adapted to apply a magnetic field that generates physical oscillations in the sensor, and a monitor adapted to detect magnetic fluctuation generated from the physical oscillations in the sensor.

3. An apparatus according to claim 1, further comprising a system adapted to monitor the sensor, the system comprising a generator adapted to apply a magnetic field that generates physical oscillations in the sensor, and a monitor adapted to detect sound waves generated from the physical oscillations in the sensor.

4. An apparatus according to claim 1, wherein the sensor comprises a metal in a form selected from at least one of: (i) a wire form, (ii) a strip form, (iii) a tubular form, and (iv) plate form.

5. An apparatus according to claim 1, wherein the sensor comprises at least one of: (i) a plurality of wires, (ii) a plurality of strips, (iii) a plurality of tubes, and (iv) a plurality of plates.

6. A method according to claim 1, further comprising coupling an anti-restenosis therapy to at least one of the stent and the sensor.

7. An apparatus according to claim 2, wherein the stent and sensor are positioned within a patient, and the system adapted to monitor the sensor is positioned outside of the patient.

8. An apparatus according to claim 2, wherein the stent and sensor are positioned within a patient, and the system adapted to monitor the sensor is positioned inside of the patient.

9. An apparatus according to claim 8, wherein the system adapted to monitor the sensor further comprising a transmitter adapted to transmit information from the system to a receiver outside of the patient.

10. An apparatus according to claim 8, wherein the system adapted to monitor the sensor is coupled to the stent.

11. An apparatus as in claim 1, wherein at least one of the stent and the sensor includes a biocompatible coating coupled thereto.

12. An apparatus comprising:
a stent adapted to be inserted into a patient, the stent having a sensor coupled thereto, the sensor comprising at least one of a magnetoelastic material and a magnetorestrictive material;
an activating coil adapted to transmit a magnetic field to the sensor;
a power supply adapted to supply power to the activating coil;
a receiving coil adapted to receive a signal from the sensor; and
a spectrometer coupled to the receiving coil.

13. A method for monitoring a patient, comprising:
positioning a stent having a sensor coupled thereto within a patient;
applying a magnetic field that generates physical oscillations in the sensor; and
detecting magnetic fluctuation generated from the physical oscillations in the sensor.

14. A method according to claim 13, wherein the sensor comprises at least one of a magnetoelastic material and a magnetorestrictive material.

15. A method according to claim 13, further comprising determining a condition of the patient based on any change in resonant frequency of the sensor over a period of time.

16. A method according to claim 15, further comprising configuring the sensor to include a plurality of individual sensors that are positioned at different locations on the stent and that are adapted to monitor different portions of the stent.

17. A method according to claim 15, further comprising initiating an action based on the condition of the patient.

18. A method according to claim 15, further comprising providing an anti-restenosis therapy coupled to at least one of the stent and the sensor.

19. A method according to claim 18, further comprising initiating the anti-restenosis therapy based on the condition of the patient.
